(19) United States
(12) Patent Application Publication
(10) Pub. No.: US 2009/0221993 A1
(43) Pub. Date: Sep. 3, 2009

(54) LASER HAIR REMOVAL DEVICE

(75) Inventors: Daniel Sohi, Essex (GB); Alexander Wolski, Gloucester (GB); Ahmad Bitter, London (GB); Tony Grant, Stroud (GB)

Correspondence Address: YOUNG & THOMPSON, 209 Madison Street, Suite 500, ALEXANDRIA, VA, 22314, US

(73) Assignee: The Dezac Group Limited, Cheltenham (GB)

(21) Appl. No.: 12/067,188

(22) PCT Filed: Sep. 20, 2006
(86) PCT No.: PCT/GB06/03470

§ 371 Date: Sep. 10, 2008

(30) Foreign Application Priority Data
Sep. 21, 2004 (GB) GB 0519252.1

(51) Int. Cl. A61B 18/20

(52) U.S. Cl. 606/3; 606/9

(57) ABSTRACT

A laser hair removal device 10 has a base unit 12 and a hand held laser wand 14. Laser 18 generates light pulses of sufficient energy and duration to damage papilla of each hair follicle in the path of the beam. The device 10 includes one or more safety features to prevent accidental misuse of the device. In particular, the device incorporates a high intensity LED 24 which makes looking at the potential path of the laser uncomfortable, thus promoting a blinking reflex. Additionally the device may incorporate a skin contact/proximity sensor 26 to prevent use of the laser away from the skin, and/or one or more locking element to prevent accidental powering of the laser.

This invention relates to laser hair removal devices and in particular, but not exclusively, to such devices intended for home usage.

In the past, cosmetic hair removal has been achieved in numerous ways including plucking, electrolysis and laser treatment. In the laser treatment process, light at a suitable wavelength (typically from about 600 nm to 900 nm) is directed towards the skin such that some of the energy is absorbed by the papilla of the hair follicle, which is damaged by the deposition of energy leading to death of the follicle and subsequent hair removal.

Because of the complexity of these devices and the safety considerations inherent with their use, traditionally laser hair removal treatment has been carried out at specialist clinics or salons rather than at home.

There is therefore a need for a laser hair removal device with inbuilt safety features so that it can be safely used by the domestic market.

Accordingly, in a broad aspect, this invention concerns a hair removal device utilising a laser generating pulses of sufficient energy and duration to damage papilla of each hair follicle in the path of the beam, with one or more safety features designed to prevent accidental misuse of the device.

In one particular aspect of this invention, there is provided a laser hair removal device for being directed towards and/or applied to the skin of a user to expose hair follicles to laser radiation for hair removal, said device comprising a body housing a hair removal laser capable of emitting radiation at a wavelength suitable for hair removal, characterised by a further light emitting element arranged to emit a high intensity beam of radiation non-injurious to the eye in generally the same direction as said hair removal laser, thereby to deter a user from aiming the device towards their eye.

The high intensity beam is non-injurious in the sense that whilst it might be unpleasant to look into the beam, no permanent injury is caused by that beam.

Preferably the high intensity beam is turned on as soon as the device is powered and before the hair removal laser is actuated. In most instances, the hair removal laser will be fired on demand using a trigger or the like. The laser will typically generate a pulsed beam of laser radiation. The light emitting element preferably emits light at a wavelength to which the eye is particularly sensitive such as light of wavelength of the order of 500-600 nm and ideally around 555 nm. The light emitting element may comprise an LED designed to provide light at the required frequency.

In another aspect, there is provided a laser hair removal device for being direct towards and/or applied to the skin of a user to expose hair follicles to laser radiation for hair removal, said device comprising a body housing a hair removal laser capable of emitting radiation at a wavelength suitable for hair removal, characterised by a filter element adjacent the end of the device to attenuate in use reflection of the beam of the hair removal laser from the skin of the user.

Preferably, the filter element comprises a tubular element extending from the end of the device around the emitted laser beam. The tubular element may conveniently be cylindrical.

In yet another aspect, the invention provides a laser hair removal device for being-directed towards and/or applied to the skin of a user to expose hair follicles to laser radiation for hair removal, said device comprising a body housing a hair removal laser capable of emitting radiation at a wavelength suitable for hair removal, characterised by a sensor for detecting contact of the device with the skin and/or proximity therewith, and control means responsive to the output of said sensor for inhibiting operation of said removal laser when the device is not in contact with or close to the skin.

The sensor may conveniently comprise two conductive contact elements and an electrical circuit for determining contact thereof with the skin. The electrical circuit may for example determine the electrical resistance. Preferably, the distance of the two contact elements is comparable in size to, or less than the size of, an eye opening to prevent firing directly into the eye. This distance is preferably in the range of about 0.5 to 1.5 cm from the centre of one element to the centre of the other element.

Other sensors could be used with similar function which determine the distance or proximity of the device to the skin. Thus, a light beam or electromagnetic radiation beam may determine the distance of the device from the skin and this output may be used by the control means to inhibit operation.

In another aspect, this invention provides a laser hair removal device for being directed towards and/or applied to the skin of a user to expose hair follicles to laser radiation for hair removal, said device comprising a body housing a hair removal laser capable of emitting radiation at a wavelength suitable for hair removal, characterised by a control means including a timer for interrupting operation of the device after a preset period.

This feature prevents overexposure of the skin which could result in burns and forces the user to remove the laser from the targeted area between uses, thereby giving sufficient time for the area to cool and also making relocation of the device onto the exact same area unlikely. In addition to the timer means, the control means may include a contact or proximity sensor of the type described above with the control means operating such that, on expiry of the predetermined period, re-energisation of the laser is allowed only after the device has been removed from and reapplied to the skin. The predetermined period will depend on a number of factors but typically may be of the order of 4 seconds.

In another aspect, this invention provides a laser hair removal device for being directed towards and/or applied to the skin of a user to expose hair follicles to laser radiation for hair removal, said device comprising a body housing a hair removal laser capable of emitting radiation at a wavelength suitable for hair removal, characterised by control means for operating said hair removal laser, said control means including a lock for preventing unauthorised use.

Said lock may take various forms; for example, it could include a keypad for the user to input an identification code, with operation being inhibited unless the correct code is input; it could include a key operated switch such that only a user with the key can turn the device on. Furthermore, the control means may inhibit operation of the laser unless a series of two or more actions are performed. For example, the control means may inhibit operation of the laser until a contact or proximity sensor detects both that the device is safely adjacent the skin- and that the user has pressed an activation switch. The control means may require that these actions are performed in the correct order.

It should be appreciated that the various functions in the above aspects may be performed by components mounted in an applicator type device which contains at least the majority of the control functionality required for operation. Alternatively, the device may be in the form of a separate base unit which contains the majority of the control circuitry with a separate head containing the laser unit and connected to the base unit by a flexible link.

Whilst the invention has been described above, it extends to any inventive combination of the features set out above, or in the following description.

The Invention may be performed in various ways, and two embodiments thereof will now be described by way of example only, reference being made to the accompanying drawings in which:—

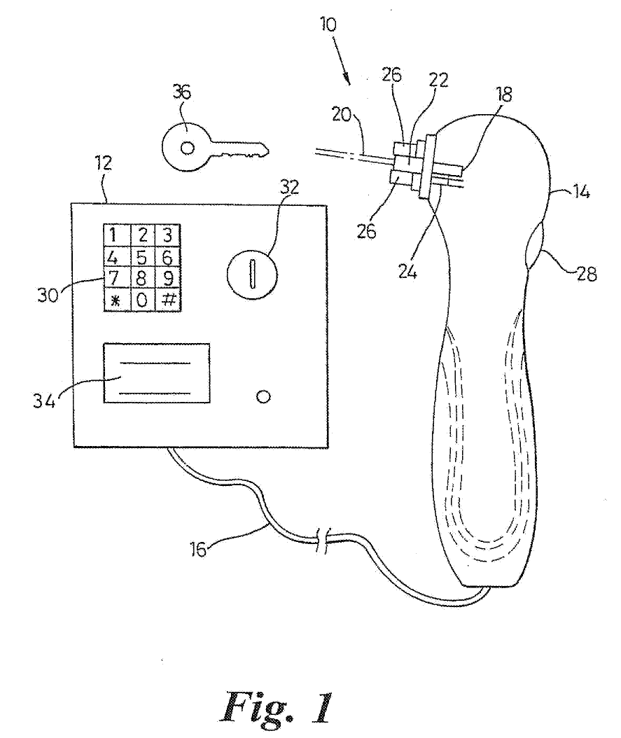

FIG. 1 is a schematic diagram of a first embodiment of a laser hair removal device in accordance with this invention;

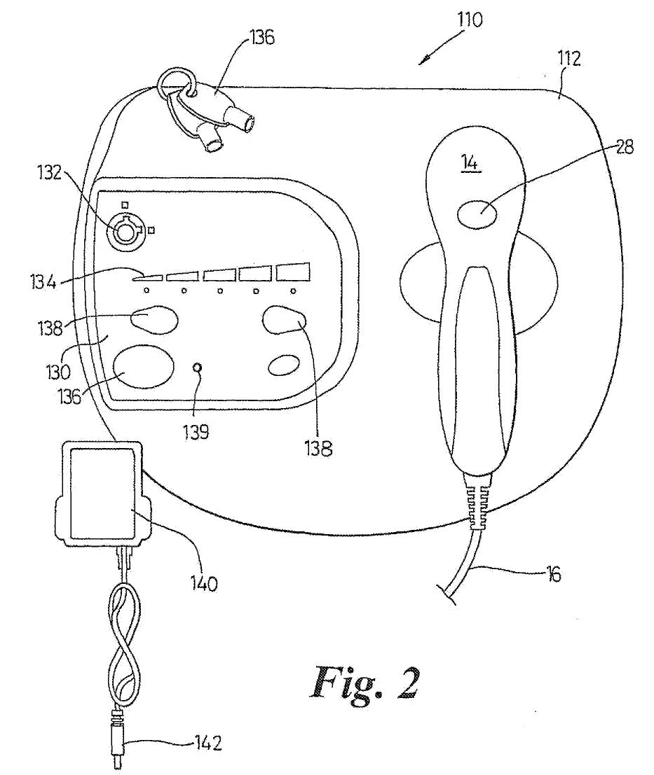

FIG. 2 illustrates a second embodiment of the a hair removal device in accordance with the invention; and

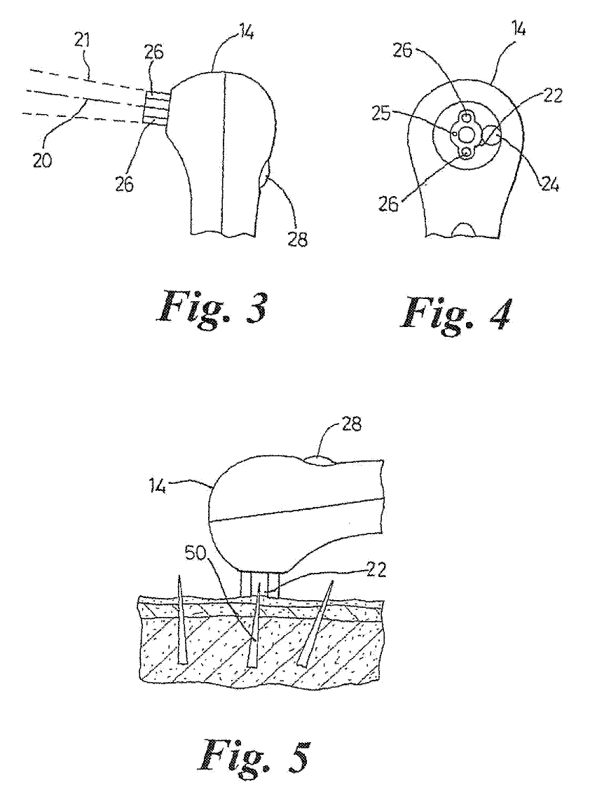

FIGS. 3,4 and 5 are views of a part of the hair removal device shown in FIG. 2.

Referring to FIG. 1, the hair removal device 10 comprises a base unit 12 and a body in the form of a laser wand 14 connected to the base unit by means of a retractable cord 16. In this particular embodiment the safety control circuitry is contained in the base unit 12 although in other embodiments all or part of this circuitry may be contained in the laser wand 14. The laser wand contains a laser 18 which produces a pulse beam of laser radiation at a wavelength selected to cause hair removal. The laser 18 is arranged to project its beam along a main axis 20. The laser 18 is housed inside the wand 14 and the beam, on leaving the main body of the wand 14 passes through a cylindrical shield 22 of semi-transparent material. Adjacent the laser 18 is a LED 24 designed also to project a high intensity beam of non-injurious visible light along substantially the same main axis 20, passing down the middle of the sleeve 22. To either side of the sleeve are provided two electrodes 26 which are connected to an electrical control circuit which detects when the laser wand 14 is in contact with the skin of a person. This is done by detecting the resistivity of the skin's surface contacted by the electrodes 26. It would be noted that, when the laser wand 14 is pressed against the skin with both contacts 26 in touch with the skin, the laser beam emitted from laser 18 is enclosed by the sleeve 22.

On the back of the wand is an operating switch 28 whose state is determined by the control circuitry in the base unit 12.

On the base unit there is an alphanumeric keypad 30 and a key operated switch 32 together with a display 34. In use, the control circuit in the base unit 12 inhibits operation of the laser 18 until certain conditions are fulfilled. In this particular example, it is necessary firstly to turn the unit on using a key 36 in the key switch 32 and then to punch in PIN code through the keypad 30. When these steps have been carried out, the control unit activates the LED 24 so that a high intensity beam of visible light is projected along the main axis 20 both as a warning of the projected axis of the laser beam from laser 18 when it is activated and also to deter users from pointing it towards their own or others' eyes. Having passed the initial security steps, the wand 14 may be offered up to the area of the skin where hair is to be removed and contact with the skin (as opposed to a non-skin surface) is detected by the control circuit using the electrodes 26. Then, and only then, can the main laser 18 be activated by pressing the operating switch 28.

It will therefore be appreciated that the embodiment illustrated in FIG. 1 requires several security stages to be passed before the laser can be operated; the first level security checks confirm the credentials of the user (i.e. being in possession of the right key and code) and thereafter physical security is assessed by ensuring that the device is actually in contact with the skin before the laser is enabled.

A second embodiment of a hair removal device is Illustrated in FIGS. 2 to 5. Features common to the first and second embodiments have like numbering. The hair removal device 110 has a base unit 112 and a hand held laser wand 14 which operate in a similar manner to the base unit and wand 12,14 described above. In this embodiment the wand 14 rests on, and is held in place by the housing 112. The base unit has a mains adapter power supply 140 which has a low voltage d.c. output plug 142 for fitting into and powering the base unit 112. Alternatively, or as well as, the base unit 122 may be powered by batteries, which may be rechargeable via the mains adapter.

In operation the base unit can be switched on by inserting key 136 into keyswitch 132. As an additional safety precaution, a predetermined series of buttons on the keypad 130 may have to be pressed to enable the laser wand 14 to be powered. The base unit has a power level indicator 134, an enable/hold button 136, power up/down button 138, and a wand enable warning light 139. The wand 14 has at least the same safety features described above.

FIGS. 3,4&5 show different views of the head of the wand 14. In FIG. 3, electrodes 26 are visible for making contact with, or coming into close proximity with, the skin of a user to enable the wand. If the laser were able to fire, the path of the coherent laser light would pass along axis 20. The beam 21 of high intensity visible but non-injurious light, which is diffuse light, travels along the generally the same axis 20. The diffuse beam 21 is of a lower intensity than the laser beam and is produced when the base 112 is powered or just before the laser operation in order to simulate an eye blinking reflex of any eye in the path of the beam 21.

FIG. 4 shows a different view of the head of the wand 14. In this embodiment there is shown a low Intensity parallel beam LED 25 which produces a low intensity narrow beam of light along the axis 20. The purpose of this light is to aim the laser so as to guide the user to the hair to be removed. This light can be switched on and can remain on before the laser is operated. A further high intensity LED 24 produces the high intensity light beam 21 for promoting blinking and this light is produced just before the laser is fired.

FIG. 5 shows the wand in use aimed, with the aid of the low intensity light from the LED 25. In this position the electrodes 26 are in contact with the skin of a user adjacent body hair 50. The base unit, once activated supplies power to the aim LED 25, to laser activation light is press, causing the high intensity LED 24 to Illuminate and then the laser to produce light for a predetermined time period, of sufficient duration to cause damage to the papilla of the hair follicle at hair 50, but not of sufficient duration to cause burning of the skin. Control at base unit 112 prevents firing of the laser again for a period sufficient to allow cooling of the user's skin.

Modifications, alternatives, variants, additions and simplifications to the embodiments described above, and Illustrated, will be readily apparent to the skilled addressee. In particular, not all the safety features mentioned above need be used together, or the features mentioned could be combined.

1-31. (canceled)

32. A laser hair removal device (10, 110) for being directed towards and/or applied to the skin of a user to expose hair follicles to laser radiation for hair removal, said device comprising a body (14) housing a hair removal laser (18) capable of emitting radiation at a wavelength suitable for hair removal, chatacterised by a further light emitting element (24) arranged to emit a high intensity beam of radiation (21) non-injurious to the eye in generally the same direction (20) as said hair removal laser, thereby to deter a user from aiming the device towards their eye.

33. A laser hair removal device as claimed in claim 32 wherein the high intensity beam (21) is turned on as soon as the device is powered or before the hair removal laser is actuated.

34. A laser hair removal device as claimed in claim 32 wherein the laser (18) generates a beam of pulsed laser radiation.

35. A laser hair removal device as claimed in claim 32 wherein the light emitting element (24) emits light (21) at a wavelength to which the eye is particularly sensitive such as light of wavelength of about 500-600 nm.

36. A laser hair removal device as claimed in claim 34 wherein the light emitting element (24) comprises an LED providing light at the required frequency.

37. A laser hair removal device (10,110) for being directed towards and/or applied to the skin of a user to expose hair follicles to laser radiation for hair removal, said device comprising a body (14) housing a hair removal laser (18) capable of emitting radiation at a wavelength suitable for hair removal, characterised by a filter element (22) adjacent the end of the device to attenuate in use reflection of the beam of the hair removal laser from the skin of the user.

38. A laser hair removal device as claimed in claim 37 wherein the filter element (22) Comprises a tubular element extending from the end of the device around the emitted laser beam.

39. A laser hair removal device (10,110) for being directed towards and/or applied to the skin of a user to expose hair follicles to laser radiation for hair removal, said device comprising a body (14) housing a hair removal laser (18) capable of emitting radiation at a wavelength suitable for hair removal, characterised by a sensor (26) or detecting contact of the device with the skin and/or proximity therewith, and control means responsive to the output of said sensor (26) for inhibiting operation of said removal laser when the device is not in contact with or close to the skin.

40. A laser hair removal device as claimed in claim 39 wherein the control means prevents reuse of the laser for a predetermined time following removal of the laser from the skin as sensed by the sensor (26).

41. A laser hair removal device (10,110) for being directed towards and/or applied to the skin of a user to expose hair follicles to laser radiation for hair removal, said device comprising a body (14) housing a hair removal laser (18) capable of emitting radiation at a wavelength suitable for hair removal, characterised by a control means including a timer for interrupting operation of the device after a preset period.

42. A laser hair removal device as claimed in claim 41, wherein the control means prevents reuse of the laser following the preset period, for a predetermined time following removal of the laser from the skin as sensed by a skin contact or skin proximity sensor (26).

43. A laser hair removal device (10,110) for being directed towards and/or applied to the skin of a user to expose hair follicles to laser radiation for hair removal, said device comprising a body (14) housing a hair removal laser (18) capable of emitting radiation at a wavelength suitable for hair removal, characterised by control means for operating said hair removal laser, said control means including a lock for preventing unauthorised use.

44. A laser hair removal device (10,110) as claimed in claim 32 wherein the body (14) of the device includes components mounted in an applicator wand which contains at least the majority of the control functionality required for operation.

45. A laser hair removal device as claimed in claim 32 wherein the device is in the form of a separate base unit (12,112) which contains the majority of the control circuitry with a separate wand (14) containing the laser unit and connected to the base unit by a flexible link (16).

46. A laser hair removal device as claimed in claim 33 wherein the laser (18) generates a beam of pulsed laser radiation.

47. A laser hair removal device as claimed in claim 35 wherein the light emitting element (24) comprises an LED providing light at the required frequency.

48. A laser hair removal device (10,110) as claimed in claim 43 wherein the body (14) of the device includes components mounted in an applicator wand which contains at least the majority of the control functionality required for operation.
